(19) United States
(12) Patent Application Publication
(10) Pub. No.: US 2024/0245898 A1
(43) Pub. Date: Jul. 25, 2024

(54) Protective Cap for Preventing Contamination of a Needle-Free Connector

(71) Applicant: Becton, Dickinson and Company, Franklin Lakes, NJ (US)

(72) Inventors: Rahul Malviya, Salmon Arm (CA); Amarsinh Jadhav, Bangalore (IN); Divik Bhargava, Meerut (IN); Aman Desai, Bengaluru (IN)

(21) Appl. No.: 18/100,220

(22) Filed: Jan. 23, 2023

(51) Int. Cl. A61M 39/16; A61M 39/20

(52) U.S. Cl. CPC A61M 39/165; A61M 39/20

(57) ABSTRACT

A protective cap engageable with a needle-free connector of an IV catheter assembly includes a collar member defining a central opening within the needle-free connector may be received and a cover member attached to the collar member so as to be pivotable or slideable relative thereto, the cover member moveable between a first position where an end connection of the needle-free connector is covered by the cover member and a second position where the end connection of the needle-free connector is uncovered from the cover member. The protective cap also includes a lever member attached to the cover member and engageable by a user to move the lever member from a non-actuated position to an actuated position. With the lever member in the non-actuated position, the cover member is in the first position, and with the lever member in the actuated position, the cover member is in the second position.

### BACKGROUND OF THE INVENTION

### Field of the Invention

The present disclosure relates generally to caps for medical connectors and, in particular, to a protective cap configured to be attached to a needle-free connector, for preventing contamination thereof.

### Description of Related Art

Catheters are commonly used to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

If not properly maintained or if exposed to a non-sterile environment, a VAD can become contaminated, sealed with blood clots, and/or can spread infection. Further, bacteria and other microorganisms may gain entry into a patient's vascular system from access hubs, ports, or valves upon connection to the VAD to deliver a fluid or pharmaceutical to a patient. Therefore, each access hub/port/valve of a VAD that is configured for attachment to a VAD is associated with some risk of transmitting a catheter related bloodstream infection (CRBSI) to a patient.

In order to decrease CRBSI cases and to ensure VADs are used and maintained correctly, many medical facilities implement sterile practices and protocols to ensure that VADs and access connectors are used properly and do not become sealed or infected. These protocols often include sterilizing the access connectors and VADs, as well as flushing the catheter with a flush solution prior to use. The sterilizing of the connector, which typically is a needle-free connector that is configured to seal off a fluid path of the VAD, may include scrubbing the needle-free connector with a sanitizing wipe before performing a subsequent flushing procedure or other desired procedure (e.g., injecting medication, performing a blood draw, locking the line, and/or capping the line). Between scrubbing of the needle-free connector and the performing of a subsequent procedure, it is necessary to allow the sanitizing solution present on the needle-free connector to dry, such that sanitizing solution does not enter into the needle-free connector when another component is connected thereto.

During the drying period for the needle-free connector, clinicians often prepare for the performing of the next procedure, such as one of the procedures mentioned above, but the clinician must also be sure to maintain the cleanliness of the needle-free connector. As prepping for a post-scrubbing procedure often requires the use of both hands, such as preparing a flushing syringe, the clinician is not able to hold-on to the needle-free connector while it is drying, and there are currently no standard procedures in place for keeping the needle-free connector clean during the period when both of the clinician hands are in use preparing for the subsequent procedure. Some non-standard practices for maintaining cleanliness of the needle-free connector include, for example, asking patient to hold the IV line, placing dirty wipes used for connector scrubbing or a new wet wipe below the connector, and/or trying to conduct subsequent procedure(s) using only one hand. Disadvantages of these non-standard practices may include not all patients being able to properly hold the connector, introducing microbes onto the connector if it rests on a used/dirty sanitizing wipe, or not allowing the connector to properly dry if it is placed on a new/wet sanitizing wipe.

It has also been known to apply a protective cap over/onto the needle-free connector when not in use, to maintain cleanliness thereof. However, existing caps are often cumbersome, may not be adaptable to different sizes/shapes of needle-free connectors, and/or may increase the difficulty of attaching another component/device of the VAD to the needle-free connector.

Accordingly, it is desired to provide a protective cap attachable to a needle-free connector that maintains the cleanliness thereof, is adaptable to different sizes/shapes of needle-free connectors, and may be easily handled or actuated by a clinician when preparing to attach another component/device to the needle-free connector.

### SUMMARY OF THE INVENTION

Provided herein is a protective cap engageable with a needle-free connector of an IV catheter assembly. The protective cap includes a collar member defining a central opening within which a portion of the needle-free connector may be received and a cover member attached to the collar member so as to be pivotable or slideable relative thereto, the cover member moveable between a first position where an end connection of the needle-free connector is covered by the cover member and a second position where the end connection of the needle-free connector is uncovered from the cover member. The protective cap also includes a lever member attached to the cover member and engageable by a user to move the lever member from a non-actuated position to an actuated position. With the lever member in the non-actuated position, the cover member is in the first position, and with the lever member in the actuated position, the cover member is in the second position.

In some embodiments, the collar member includes a friction pad positioned on an inner surface of the collar member configured to secure the collar member on the portion of the needle-free connector

In some embodiments, the collar member includes a first collar half including a buckle receptacle and a second collar half including a buckle stem, wherein the first collar half is joinable to the second collar half via insertion of the buckle stem into the buckle receptacle, with positioning of the buckle stem relative to the buckle receptacle being adjustable, such that a diameter of the central opening of the collar member is also adjustable.

In some embodiments, the protective cap includes a hinge connecting the cover member to the collar member, the hinge positioned at a top edge of the collar member, with the cover member pivoting relative to the collar member via the hinge to move between the first position and the second position.

In some embodiments, the lever member is a downward oriented lever arranged approximately parallel to an outer surface of the collar member, the lever member being pullable downward to move the lever member from the non-actuated position to the actuated position, thereby pivoting the cover member from the first position to the second position.

In some embodiments, the lever member is a generally horizontally oriented lever extending radially outward from the cover member, the lever member being pushable downward to move the lever member from the non-actuated position to the actuated position, thereby pivoting the cover member from the first position to the second position.

In some embodiments, the lever member is a V-shaped push lever linkage attached to the cover member at a first linkage end and to the collar member at a second linkage end, the lever member being pressable radially inward to move the lever member from the non-actuated position to the actuated position, thereby pivoting the cover member from the first position to the second position.

In some embodiments, the hinge is biased to return the cover member from the second position to the first position, upon termination of a pushing force on the lever member.

In some embodiments, the collar member includes a pair of restraint arms extending upwardly past a tope edge of the collar member, each restraint arm of the pair of restraint arms comprising a hook member at a distal end thereof, and wherein the cover member comprises tracks formed thereon with which the hook member of each restraint arm is slidingly engaged.

In some embodiments, the lever member is an angled lever extending downwardly and outwardly from the cover member, the lever member being pressable radially inward toward the collar member by a user to move the lever member from the non-actuated position to the actuated position, thereby sliding the cover member from the first position to the second position, via sliding of the hook member of each restraint arm along the tracks.

In some embodiments, the cover member includes a first cover portion and a second cover portion and the lever member includes a first lever and a second lever, with the first lever attached to the first cover portion and the second lever attached to the second cover portion.

In some embodiments, the protective cap includes a first fulcrum positioned on a first side of the collar member and a second fulcrum positioned on a second side of the collar member, with the first lever attached to the first cover portion at the first fulcrum and the second lever attached to the second cover portion at the second fulcrum, such that the first lever and the first cover portion pivot about the first fulcrum and the second lever and the second cover portion pivot about the second fulcrum.

In some embodiments, the protective cap includes a first bi-stable hinge connecting the first fulcrum feature to the collar member and a second bi-stable hinge connecting the second fulcrum feature to the collar member, the first bi-stable hinge and the second bi-stable hinge positioned on opposing first and second sides of the collar member, with the first cover portion and the second cover portion pivoting relative to the collar member via the first bi-stable hinge and the second bi-stable hinge, respectively, to move between the first position and the second position.

In some embodiments, the protective cap includes a first hinge connecting the first cover portion to the collar member and a second hinge connecting the second cover portion to the collar member, the first hinge and the second hinge positioned on a top side of the collar member, with the first cover portion and the second cover portion pivoting relative to the collar member via the first hinge and the second hinge, respectively, to move between the first position and the second position.

In some embodiments, each of the first lever and the second lever is pressable radially inward to move the first lever and the second lever from the non-actuated position to the actuated position, thereby pivoting each of the first cover portion and the second cover portion from the first position to the second position.

In some embodiments, the first bi-stable hinge and the second bi-stable hinge are configured to hold the first cover portion and the second cover portion in the second position upon termination of a radially inward directed pushing force on the first lever and the second lever.

In some embodiments, the lever member is engageable by a single hand of the user to move the lever member from the non-actuated position to the actuated position.

Also provided is an IV catheter assembly including a catheter adapter, a catheter coupled to the catheter adapter and extending out distally therefrom, so as to be positionable intravenously within a patient, an extension line coupled to the catheter adapter and extending out proximally therefrom, a luer hub positioned at a proximal end of the extension line and in fluid communication with the catheter through the extension line and the catheter adapter, and a needle-free connector having a distal connector end and a proximal connector end, with the distal connector end coupled to the luer hub. The IV catheter assembly also includes a protective cap coupled to the proximal connector end of the needle-free connector, with the protective cap including a collar member defining a central opening within which a portion of the needle-free connector may be received, a cover member attached to the collar member so as to be pivotable or slideable relative thereto, the cover member moveable between a first position where an end connection of the needle-free connector is covered by the cover member and a second position where the end connection of the needle-free connector is uncovered from the cover member, and a lever member attached to the cover member and engageable by a user to move the lever member from a non-actuated position to an actuated position. With the lever member in the non-actuated position, the cover member is in the first position, and with the lever member in the actuated position, the cover member is in the second position.

In some embodiments, the proximal connector end of the needle-free connector is a female luer connection including a tapered cavity and a threaded outer surface.

In some embodiments, the collar member is positioned about the needle-free connector at a location thereon distal from the threaded outer surface, with the tapered cavity and threaded outer surface being uncovered from the protective cap when the protective cap is in the first position and being covered by the protective cap when the protective cap is in the second position.

### BRIEF DESCRIPTION OF THE DRAWINGS

FIG. 1 is a perspective view of an IV catheter assembly, according to an aspect of the present disclosure;

FIGS. 2A and 2B illustrate an exemplary needle-free connector, as is known in the prior art;

FIGS. 3A and 3B are perspective views of a protective cap in a closed position and an open position, respectively, according to an aspect of the disclosure;

FIGS. 4A and 4B illustrate the protective cap of FIGS. 3A and 3B, relative to a needle-free connector to which the protective cap is coupled;

FIG. 5 is a front view of a protective cap, when positioned on a needle-free connector, according to another aspect of the disclosure;

FIGS. 6A and 6B are front views of a protective cap in a closed position and an open position, respectively, when positioned on a needle-free connector, according to another aspect of the disclosure;

FIGS. 7A and 7B are front views of a protective cap in a closed position and an open position, respectively, when positioned on a needle-free connector, according to another aspect of the disclosure;

FIG. 8 is a perspective view of a protective cap, when positioned on a needle-free connector, according to another aspect of the disclosure;

FIGS. 9A and 9B are front views of a protective cap in a closed position and an open position, respectively, according to an aspect of the disclosure;

FIGS. 10A and 10B illustrate the protective cap of FIGS. 9A and 9B, relative to a needle-free connector to which the protective cap is coupled;

FIG. 11 is a front view of a protective cap, when positioned on a needle-free connector, according to another aspect of the disclosure;

FIG. 12 is a front view of a protective cap, when positioned on a needle-free connector, according to another aspect of the disclosure; and

FIGS. 13A and 13B are front views of a protective cap in a closed position and an open position, respectively, when positioned on a needle-free connector, according to another aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with another component or with a patient. Thus, for example, the end of a device first contacting another component or the body of the patient would be the distal end, while the opposite end of the device being manipulated by the user would be the proximal end of the device.

The terms “first”, “second”, and the like are not intended to refer to any particular order or chronology, but refer to different conditions, properties, or elements.

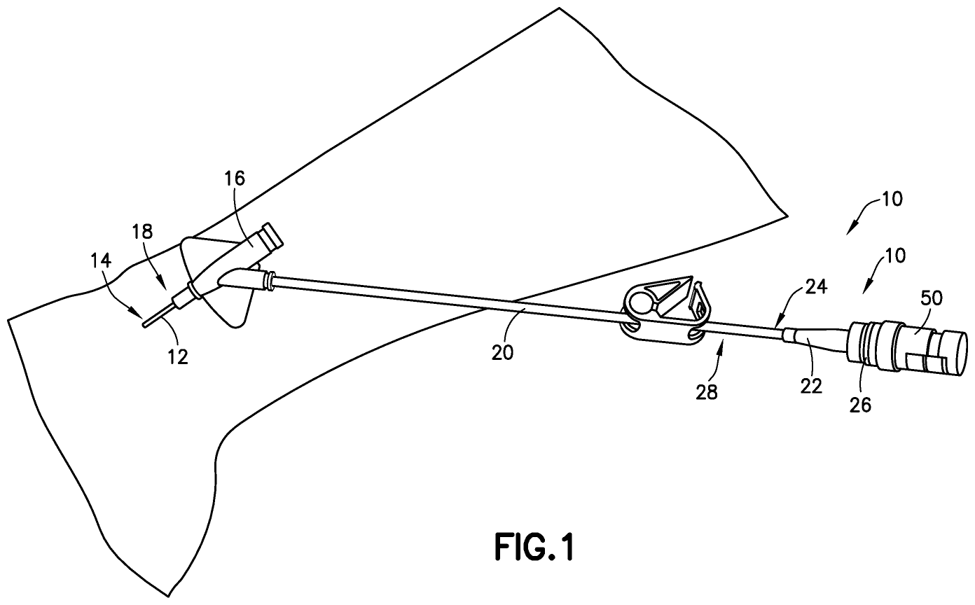

Reference is first made to FIG. 1, which depicts an intravenous (IV) catheter assembly 10 with which aspects of the disclosure may be implemented, according to a non-limiting embodiment. The IV catheter assembly 10 includes a catheter tube 12 having a distal end 14 that may be inserted transcutaneously through the skin of a patient at an insertion site. The IV catheter assembly 10 also includes a catheter adapter (or hub) 16 coupled to a proximal end 18 of the catheter tube 12, with the adapter 12 shown in FIG. 1 as including a side port to which an extension leg 20 is connected and extends out proximally from, according to one embodiment.

The extension leg 20 includes a luer hub 22 positioned at a proximal end 24 thereof. As used herein, a “luer” hub refers to a connector that includes a tapered portion (i.e., a luer taper) for creating a friction engagement between a tapered stem or elongated member of a male luer connection and a tapered cavity. The luer hub 22 may be configured as a female luer connection having a tapered cavity (not shown) configured to receive and engage a tapered stem or elongated member of a male luer connection, as well as a threaded outer surface (not shown) configured to engage threads on the inner surface of the annular shield of a male luer connection. Coupled to the luer hub 22 is a needle-free connector 26 that seals off the luer hub 22 and fluid path 28 (through luer hub 22, extension leg 20, catheter adapter 16, and catheter tube 12) of the IV catheter assembly 10.

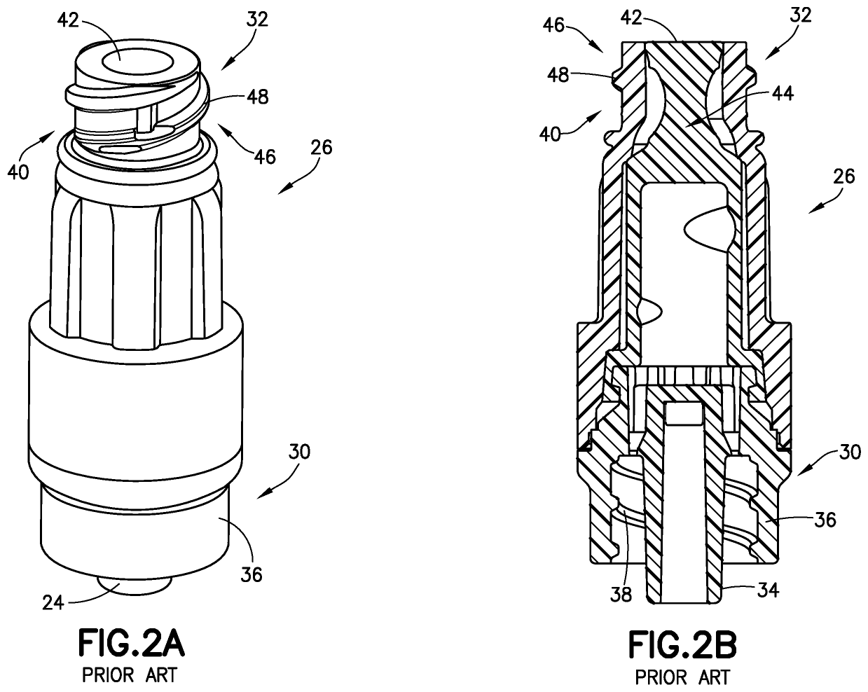

A known needle-free connector 28 that may be connected to the luer hub 22 of the IV catheter assembly 10 is shown in FIGS. 2A and 2B. The needle-free connector 26 may generally include a male luer connection 30 and a female luer connection 32. As used herein, a “luer connector” refers to a connector that includes a tapered portion (i.e., a luer taper) for creating a friction engagement between a tapered stem or elongated member and a tapered cavity. As regards use of the needle-free connector 26 with the IV catheter assembly 10 of FIG. 1, the male luer connection 30 of needle-free connector 26 may be connected to the luer hub 22, while the female luer connection 32 may be connected to a device or component useable with the IV catheter assembly 10, such as a syringe or other fluid transfer device (e.g., blood draw device).

The male luer connection 30 of needle-free connector 26 includes a tapered stem 34 or elongated member having a tapered outer surface. The tapered stem 34 may be received by a corresponding tapered cavity of a female luer connection. The male luer connection 30 can also include an annular shield 36 extending about the tapered stem 34 that includes threads 38 on an inner surface thereof configured to engage corresponding threads on an outer surface of a female luer connection.

The female luer connection 32 of needle-free connector 26 includes an elongated proximal end portion 40 with a cover or septum 42 (including a slit) positioned over an opening of a tapered cavity 44 configured to receive and engage a corresponding tapered stem or elongated member of a male luer connection. The female luer connection 32 can also include an outer surface 46 that includes threads 48 configured to engage corresponding threads on an inner surface of an annular shield of a male luer connection.

Referring again now to FIG. 1, a protective cap 50 may be coupled to the needle-free connector 26 to protect portions of the connector and selectively provide access to the connector, as desired by a clinician. Specifically, the protective cap 50 may be positioned over the female luer connection 32 (FIGS. 2A and 2B) of the needle-free connector 26. The protective cap 50 is configured such that it may be actuated from a first position to a second position. When the needle-free connector 26 is not in use (i.e., when no component/device is connected thereto), the protective cap 50 may be in the first position, such that the protective cap 50 covers the female luer connection 32, to protect the connection and prevent the connection from being contaminated by, for example, microbes, debris, or other contaminants. When it is desired to connect a component/device to the needle-free connector 26, the protective cap 50 may be moved to the second position, such that the female luer connection 32 is uncovered and accessible to connect a component/device thereto. Beneficially, and as will be explained in further detail below, the protective cap 50 is configured such that it may be actuated between the first and second positions using a single-hand actuation technique or motion, such that the other hand of a user (e.g., clinician) may remain free to perform other tasks.

The protective caps 50 of the present disclosure are configured to engage a variety of different configurations and orientations of needle-free connectors. As will be appreciated by those skilled in the art, there are numerous different commercially available needle-free connectors, which include different variations of male or female luer connections. The protective caps 50 of the present disclosure are configured to adapt so that they can be secured to numerous different types and sizes of female luer connections. For example, the protective caps 50 of the present disclosure are configured to attach to female luer connections of various needle-free connectors, including without limitation, the BD Q-Syte™, BD MaxZero™, BD MaxPlus™, and SmartSite™ needle free connectors by Becton Dickinson and Company, as well as MicroClave® connectors (ICU Medical Inc.) and Ultrasite® or Caresite® connectors (B. Braun Medical Inc.).

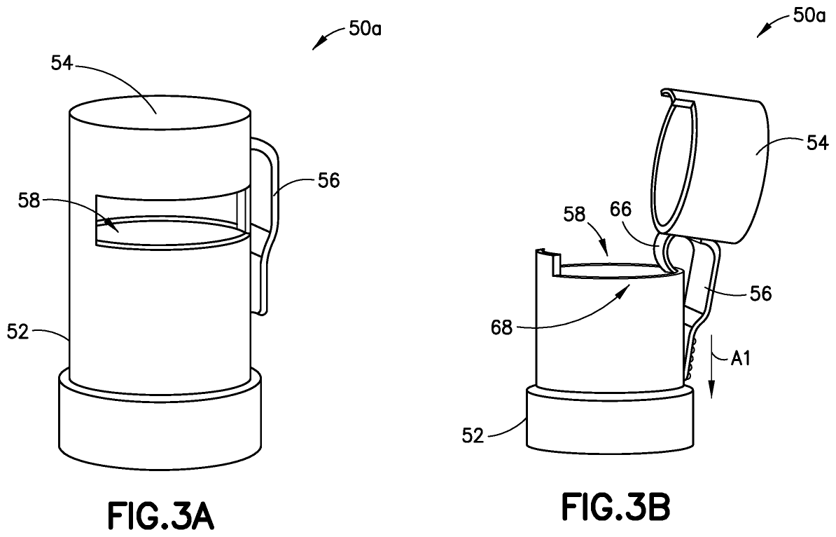

FIGS. 3-13 illustrate embodiments of protective caps 50 useable with a needle-free connector, in accordance with aspects of the disclosure. The protective caps 50 may, for example, mate with the female luer connection 32 of the needle-free connector 26 in the IV catheter assembly 10 of FIG. 1.

Referring first to FIGS. 3-5, a protective cap 50a is shown that may be attached to a female luer connection end of a needle-free connector 26 (i.e., the proximal end of the needle-free connector 26, in FIG. 1). The protective cap 50a generally includes a collar member 52, a cover member 54 and a lever member 56 that, in some embodiments, may be integrally formed with each other as part of a single/unitary protective cap 50a. According to embodiments, the protective cap 50a may be formed from a thermoplastic polymer material, such as polyester, polycarbonate, polypropylene, polyethylene, polyethylene terephthalate, or acrylonitrile butadiene styrene, such that the protective cap 50a may be formed via an injection molding process, for example.

The collar member 52 may be formed as a generally cylindrical member that defines a central opening 58 therethrough, within which a portion of the needle-free connector 26 may be received, so as to seat the protective cap 50a on the needle-free connector 26. The height of the collar member 52 is such that, when the protective cap 50a is seated on the needle-free connector 26, the collar member 52 does not extend up to the threaded outer portion 46 of the female luer connection 32 on needle-free connector 26.

In one embodiment, and as shown in FIG. 3A, the collar member 52 includes a friction pad or pads 60 positioned on an inner surface thereof. The friction pad(s) 60 is positioned to engage with an outer surface of the needle-free connector 26 when the protective cap 50a is seated on the needle-free connector 26, so as to aid in retaining the protective cap 50a on the needle-free connector 26.

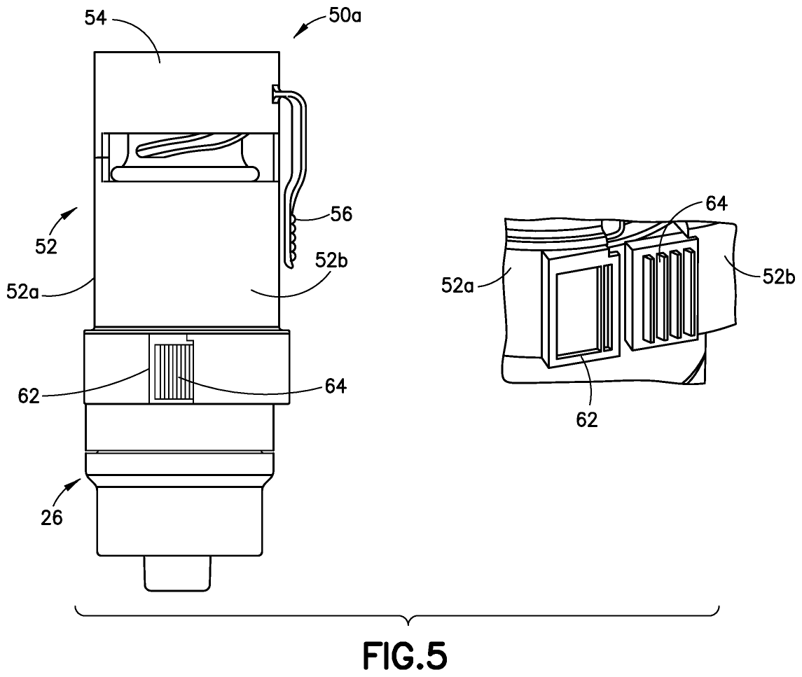

In another embodiment, and as shown in FIG. 5, the collar member 52 is configured as a split member that includes a first collar half 52a and a second collar half 52b that are separated but joinable together to position the collar member 52 about the needle-free connector 26. The first collar half 52a includes a buckle receptacle 62 thereon, while the second collar half 52b includes a buckle stem 64 thereon—with the first collar half 52a being joined to the second collar half 52b via insertion of the buckle stem 64 into the buckle receptacle 62. In some embodiments, the buckle stem 64 includes a plurality of graduations or bumps thereon that function to selectively control an amount by which the buckle stem 64 is inserted into the buckle receptacle 62, with the amount by which the buckle stem 64 is inserted into the buckle receptacle 62 controlling/adjusting the size of the central opening 58 defined by the collar portion 52—such that the protective cap 50a may be seated on needle-free connectors of various size/diameter.

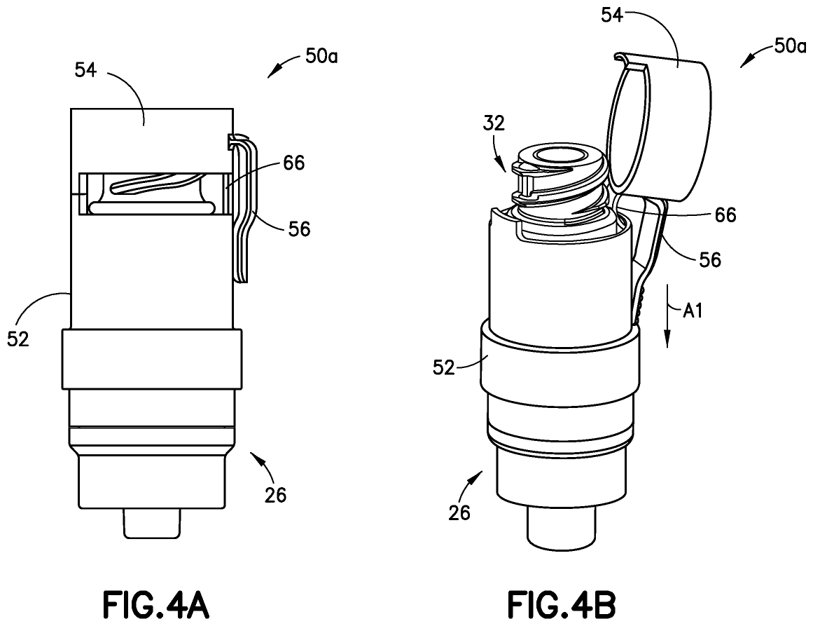

As best shown in FIGS. 3B and 4B, the cover member 54 comprises a generally (inverted) cup-shaped member that is joined to the collar member 52 by way of a flexible hinge 66. The hinge 66 is provided at a top edge 68 of the collar member 52, along one side thereof, and enables the cover member 54 to be rotated or pivoted relative to the collar member 52. Specifically, the hinge 66 enables the cover member 54 to pivot from a closed or “first” position (FIGS. 3A and 3B) where the cover member 54 substantially encloses or covers the female luer connection 32 (i.e., septum, 42, tapered cavity 44, and threads 48) of needle-free connector 26 to an open or “second” position (FIGS. 3B and 4B) where the female luer connection 32 is uncovered and accessible for connecting a male luer connection of another component/device thereto.

The lever member 56 is attached to the cover member 54 and is configured so as to be engageable by a user to move the lever member 56 from a non-actuated position to an actuated position. In the embodiment of FIGS. 3-5, the lever member 56 is attached to one side of the cover member 54 and extends downward therefrom in a direction generally parallel to an outer surface of the collar member 52. A far end of the lever member 56 is provided as a free end that may be grasped by a user, with the lever member 56 having some flexibility to so as to be separable away from the cover member 54 and collar member 52 by a small amount to enable a user to properly grab the lever member 56. The lever member 56 is configured so as to be grasped and pulled downward by a user, as indicated by arrow A1, via a single hand actuation, to move the lever member 56 from its non-actuated position to its actuated position. Movement of the lever member 56 from its non-actuated position to its actuated position causes a corresponding movement of the cover member 54 from its first position to its second position, thereby uncovering the female luer connection 32 of the needle-free connector 26.

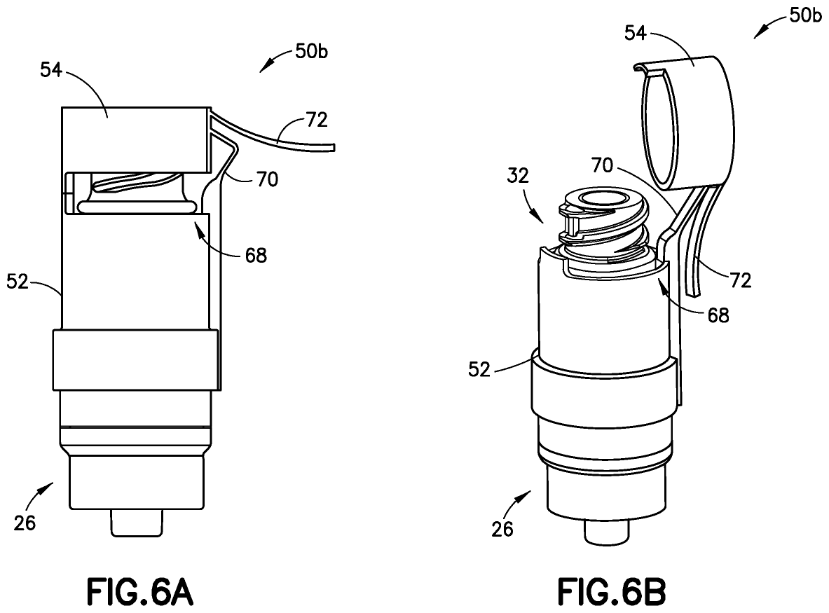

Referring now to FIGS. 6A and 6B, a protective cap 50b is shown according to another aspect of the disclosure. The protective cap 50b is substantially similar to that of protective cap 50a shown in FIGS. 2-5 (i.e., collar member 52 and cover member 54 are the same), and thus like members are labeled identically in protective cap 50b. As provided in detail here below, a hinge 70 and lever member 72 are provided on protective cap 50b that are structured differently than the hinge 66 and lever member 56 of FIGS. 2-6, and provides another means for pivoting cover member 54 from the first position to the second position.

As shown in FIGS. 6A and 6B, a hinge 70 is provided on protective cap 50b adjacent the top edge 68 of the collar member 52, along one side thereof, that enables the cover member 54 to be rotated or pivoted relative to the collar member 52 between a first position and a second position, as previously described in FIGS. 3-5. The hinge 70 is shaped and structured to bias the cover member 54 to the first position, absent any opposing force being applied to retain the cover member 54 in the second position.

The lever member 72 of protective cap 50b is attached to the cover member 54 and is configured so as to be engageable by a user to move the lever member 72 from a non-actuated position to an actuated position. In the embodiment of FIGS. 6A and 6B, the lever member 72 is attached to one side of the cover member 54 and extends radially outward from the cover member 54—with the lever member 72 having a generally horizontal orientation when the cover member 54 is in the first position. The lever member 72 is configured so as to be pushed in a downward direction by a user, via a single hand actuation, to move the lever member 72 from its non-actuated position to its actuated position. Movement of the lever member 72 from its non-actuated position to its actuated position causes a corresponding movement of the cover member 54 from its first position to its second position, thereby uncovering the female luer connection 32 of the needle-free connector 26. The cover member 54 may be retained in its second position as long as the lever member 72 is depressed by a user but, as described above, the cover member 54 will return from the second position to the first position upon termination of a pushing force on the lever member 72, due to the biasing of the hinge 70.

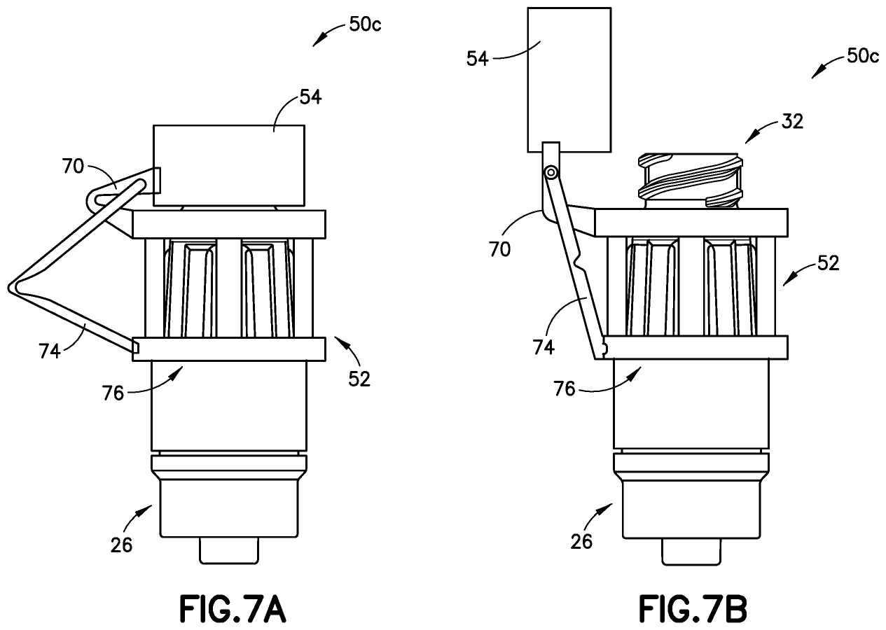

A protective cap 50c is shown according to another aspect of the disclosure in FIGS. 7A and 7B. The protective cap 50c is substantially similar to that of protective cap 50b shown in FIGS. 6A and 6B regarding the general structure of the collar member 52 (although collar member 52 is provided as a cage-type structure in cap 50c versus a solid cylindrical structure in cap 50b), cover member 54, and hinge 70 (i.e., with the hinge 70 being shaped and structured to bias the cover member 54 to the first position), and thus like members are labeled identically in protective cap 50c. However, as shown in FIGS. 7A and 7B, a lever member 74 provided on protective cap 50c to move the cover member 54 is provided as a V-shaped push lever linkage. The V-shaped push lever linkage 74 is attached at a first end thereof to the hinge 70 (on opposing sides of the hinge 70, at a position on hinge 70 adjacent cover member 54), and at a second end thereof to the collar member 52 (adjacent a bottom edge 76 of the collar member 52). The V-shaped push lever linkage 74 is configured so as to be pushed in a radially inward direction (toward the collar member 52) by a user, via a single hand actuation, to move the push lever linkage 74 from its non-actuated position to its actuated position—with the V-shaped push lever linkage 74 being deflected into a generally straight configuration responsive to the pushing force applied thereto. The inward pushing and resulting straightening of the push lever linkage 74 into its actuated position causes a corresponding pivoting of the hinge 70 and cover member 54 to occur, with the cover member 54 pivoting from its first position to its second position and thereby uncovering the female luer connection 32 of the needle-free connector 26. The cover member 54 may be retained in its second position as long as the push lever linkage 74 is depressed by a user but, as described above regarding the cap 50b of FIGS. 6A and 6B, the cover member 54 will return from the second position to the first position upon termination of a pushing force on the push lever linkage 74, due to the biasing of the hinge 70.

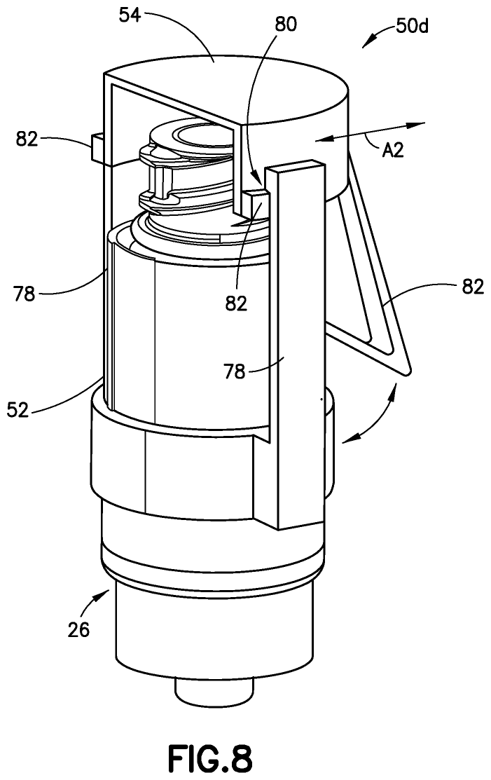

Referring now to FIG. 8, a protective cap 50d is shown in accordance with another embodiment of the disclosure. The protective cap 50d differs from the protective caps 50a, 50b, 50c shown in FIGS. 3-7, in that the cover member 54 thereof is moved between a first (closed) position and a second (open) position via a sliding of the cover member 54 relative to the collar member 52, rather than a pivoting of the cover member 54.

To enable a sliding movement of the cover member 54 relative to the collar member 52, a pair of restraint arms 78 is provided on the collar member 52. The restraint arms 78 are offset radially outward from the main body of the collar member 52 and extend upwardly past a top edge 68 of the collar member 52, with an end of the restraint arms 78 vertically aligned with, for example, a vertical mid-point of the cover member 54. Each restraint arm 78 includes a downward oriented hook feature 80 formed thereon, and the hook feature 80 on each of restraint arms 78 interacts with a track or tracks 82 formed on the cover member 54. The track(s) 82 provided on the cover member 54 jut radially outward from the main body of the cover member 54, such that the hook features 80 of the restraint arms 78 are positioned to ride along the tracks 82.

A lever member 84 is attached to the cover member 54 and is configured so as to be engageable by a user to move the lever member 84 from a non-actuated position to an actuated position. In the embodiment of FIG. 8, the lever member 84 is provided as an angled lever attached to one side of the cover member 54, with the angled lever extending downwardly and outwardly from the cover member 54. A far end of the lever member 84 is provided as a free end that may be pressed radially inward and toward the collar member 52 by a user, via a single hand actuation, to move the lever member 84 from its non-actuated position to its actuated position. Movement of the lever member 84 from its non-actuated position to its actuated position causes a corresponding sliding movement of the cover member 54 relative to the collar member 52 (i.e., via sliding of hook features 80 along tracks 82), as indicated by arrow A2, with the cover member 54 sliding from its first position to its second position, thereby uncovering the female luer connection 32 of the needle-free connector 26.

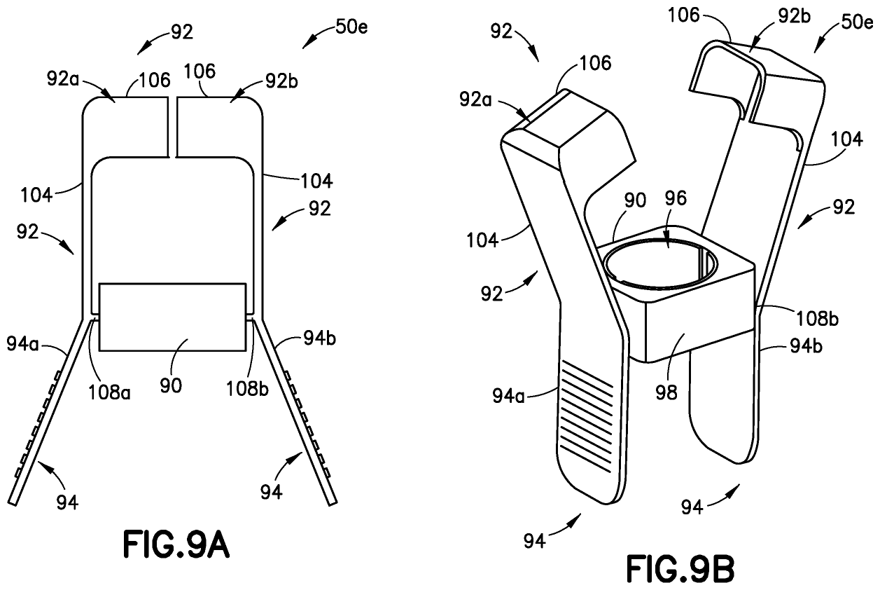

Referring now to FIGS. 9-11, a protective cap 50e is shown in accordance with another embodiment of the disclosure. The protective cap 50e is configured as a clamp-type cap that may be attached to a female luer connection end of a needle-free connector 26 (i.e., the proximal end of the needle-free connector 26, in FIG. 1). The protective cap 50e generally includes a collar member 90, a cover member 92 and a lever member 94 that, in some embodiments, may be integrally formed with each other as part of a single/unitary protective cap 50c. According to embodiments, the protective cap 50e may be formed from a thermoplastic polymer material, such as polyester, polycarbonate, polypropylene, polyethylene, polyethylene terephthalate, or acrylonitrile butadiene styrene, such that the protective cap 50c may be formed via an injection molding process, for example.

The collar member 90 may have a generally square-shaped profile, with a central opening 96 being defined that extends through the collar member 90 within which a portion of the needle-free connector 26 may be received, so as to seat the protective cap 50e on the needle-free connector 26. The height of the collar member 90 is such that, when the protective cap 50e is seated on the needle-free connector 26, the collar member 90 does not extend up to the threaded outer portion 46 of the female luer connection 32 on needle-free connector 26.

In one embodiment, and as shown in FIG. 9B, the collar member 90 includes a friction pad or pads 98 positioned on an inner surface the thereof. The friction pad(s) 98 is positioned to engage with an outer surface of the needle-free connector 26 when the protective cap 50e is seated on the needle-free connector 26, so as to aid in retaining the protective cap 50e on the needle-free connector 26.

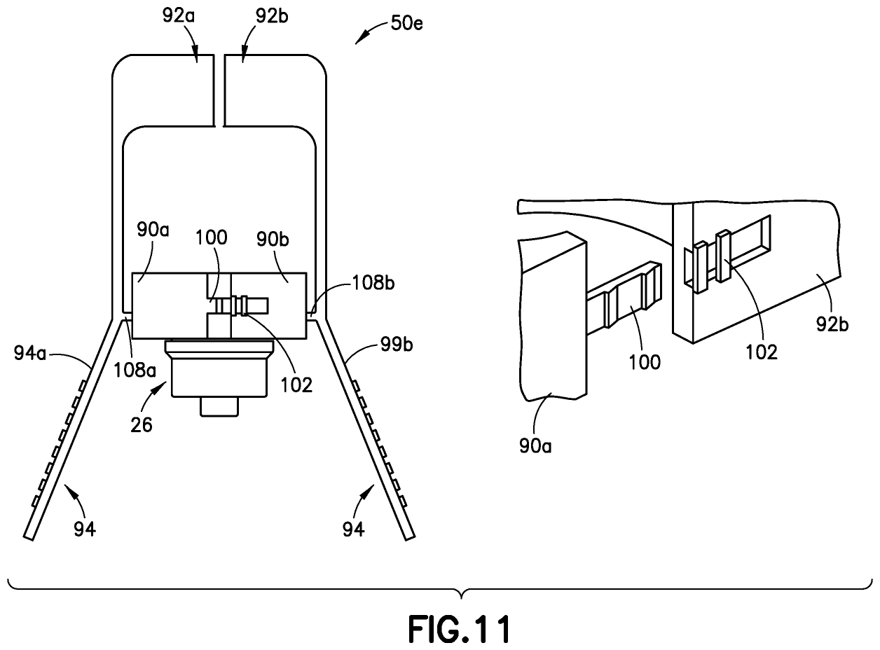

In another embodiment, and as shown in FIG. 11, the collar member 90 is configured as a split member that includes a first collar half 90a and a second collar half 90b that are separated but joinable together to position the collar member 90 about the needle-free connector 26. The first collar half 90a includes a buckle stem 100 thereon, while the second collar half 90b includes a buckle receptacle 102 thereon—with the first collar half 90a being joined to the second collar half 90b via insertion of the buckle stem 100 into the buckle receptacle 102. In some embodiments, the buckle stem 100 includes a plurality of graduations or bumps thereon that function to selectively control an amount by which the buckle stem 100 is inserted into the buckle receptacle 102, with the amount by which the buckle stem 100 is inserted into the buckle receptacle 102 controlling/adjusting the size of the central opening 96 defined by the collar member 90—such that the protective cap 50e may be seated on needle-free connectors of various size/diameter.

As shown in FIGS. 9-11, the cover member 92 of protective cap 50e is configured as a clamp-type cover that includes a first cover portion 92a and a second cover portion 92b. Each of the first cover portion 92a and the second cover portion 92b may generally be described as having an inverted L-shape, with a linear arm 104 extending generally upward to an overhanging cover 106. The linear arm 104 of the first cover portion 92a is positioned adjacent a first side of the collar member 90, while the linear arm 104 of the second cover portion 92b is positioned adjacent the opposing second side of the collar member 90. The overhanging cover 106 of each of the first cover portion 92a and second cover portion 92b then extend radially inward from their respective arm 104, with the overhanging covers 106 from the first cover portion 92a and second cover portion 92b coming into contact (or nearly into contact) with each other to form a cover over the female luer connection 32 of the needle-free connector 26.

Similar to the cover member 92, the lever member 94 of protective cap 50e includes a first lever 94a and a second lever 94b. The first lever 94a is attached to (i.e., integrally formed with) the first cover portion 92a and the second lever 94b is attached to (i.e., integrally formed with) the second cover portion 92b, with the levers 94a, 94b attached to the cover portions 92a, 92b so as to extend downwardly and outwardly from a bottom end thereof (i.e., from a bottom end of the linear arm 104). Each of the first lever 94a and the second lever 94b is configured so as to be engageable by a user to move the lever 94a, 94b from a non-actuated position to an actuated position.

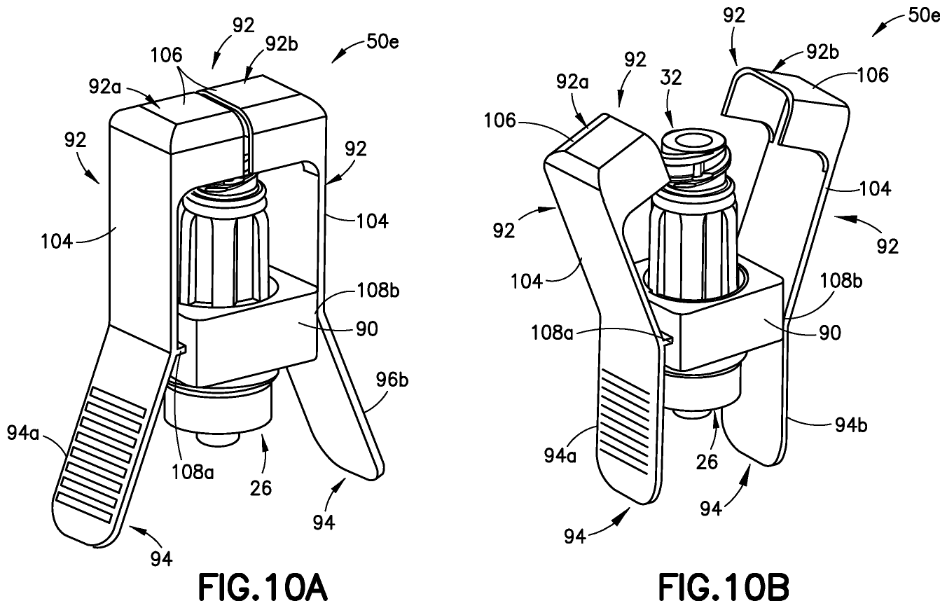

The first lever 94a and first cover portion 92a are joined to the collar member 90 via a first fulcrum feature 108a provided on the first side of the collar member 90, while the second lever 94b and second cover portion 92b are joined to the collar member 90 via a second fulcrum feature 108b provided on the second side of the collar member 90. With the first lever 94a joined to the first cover portion 92a at the first fulcrum feature 108a, the integrally formed first lever 94a and first cover portion 92a is configured to pivot about the first fulcrum feature 108a. Similarly, with the second lever 94b joined to the second cover portion 92b at the second fulcrum feature 108b, the integrally formed second lever 94b and second cover portion 92b is configured to pivot about the second fulcrum feature 108b. The pivoting of the cover portions 92a, 92b relative to their respective fulcrum features 108a, 108b enables the cover portions 92a, 92b to pivot from a closed or “first” position (FIGS. 9A and 10A) where the cover portions 92a, 92b substantially enclose or cover the female luer connection 32 (i.e., the septum 42, tapered cavity 44, and threads 48) to an open or “second” position (FIGS. 9B and 10B) where the female luer connection 32 is uncovered and accessible for connecting a male luer connection of another component/device thereto.

In operation of the protective cap 50e, each of the first lever 94a and the second lever 94b is pressed radially inward by a user to move the first lever 94a and the second lever 94b from their non-actuated position to their actuated position. Movement of the first and second levers 94a, 94b from their non-actuated position to their actuated position causes a corresponding pivoting of the first and second cover portions 92a, 92b about the fulcrum features 108a, 108b, with the first cover portion 92a and the second cover portion 92b pivoting and separating from the first position to the second position, thereby uncovering the female luer connection 32 of the needle-free connector 26.

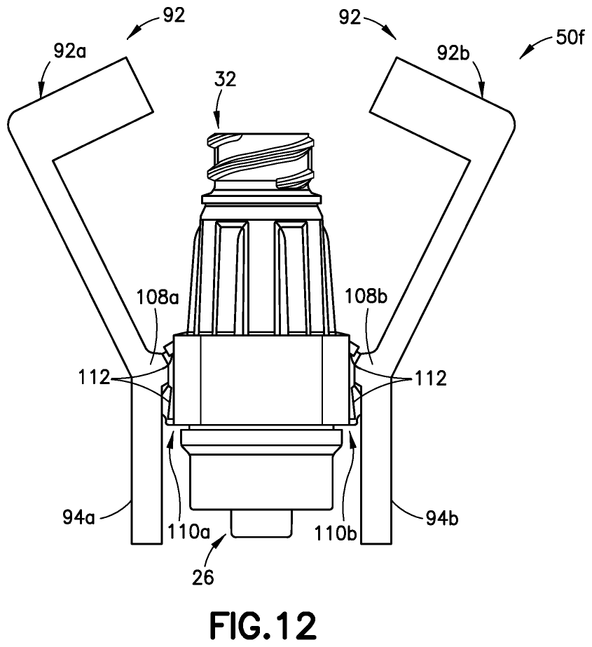

Referring now to FIG. 12, a protective cap 50f is shown in accordance with another embodiment of the disclosure. The protective cap 50f is substantially similar to that of protective cap 50e shown in FIGS. 9-11, with the collar member 90, the cover portions 92a, 92b, and the levers 94a, 94b being the same, except that bi-stable compliant hinge mechanisms 110a, 110b are further included in the protective cap 50f via which the fulcrum features 108a, 108b are joined to the collar member 90.

As shown in FIG. 12, a bi-stable compliant hinge mechanism 110a, 110b is provided on each of the first side of the collar member 90 and the second side of the collar member 90. Each bi-stable compliant hinge mechanism 110a, 110b includes a pair or arms 112—with a first arm and a second arm of each hinge mechanism 110a, 110b joined to opposing sides of the fulcrum feature 108a, 108b to which it is connected. Each of the arms 112 of the hinge mechanism 110a, 110b is a deflectable member, and the arms 112 work collectively to balance forces such that the first and second cover portions 92a, 92b can be retained in either the first position or the second position without a continued application of force to the levers 94a, 94b by a user. That is, the bi-stable compliant hinge mechanism is stable with the levers 94a, 94b either fully in the non-actuated position or fully in the actuated position, such that the first and second cover portions 92a, 92b will remain in their present position when the levers 94a. 94b are either fully in the non-actuated position or fully in the actuated position. As pertains to the first and second cover portions 92a, 92b remaining in their second position, this means that with the levers 94a, 94b in their actuated position, the first and second cover portions 92a, 92b will remain in the second position even when a user is no longer applying a radially inward-directed pushing force to the levers 94a, 94b. Accordingly, the protective cap 50f eliminates the need for a user to apply a continuous force to maintain the cover portions 92a, 92b in the open second position, thereby allowing the user to focus their efforts on connecting an external component/device to the female luer connection 32 of needle-free connector 26.

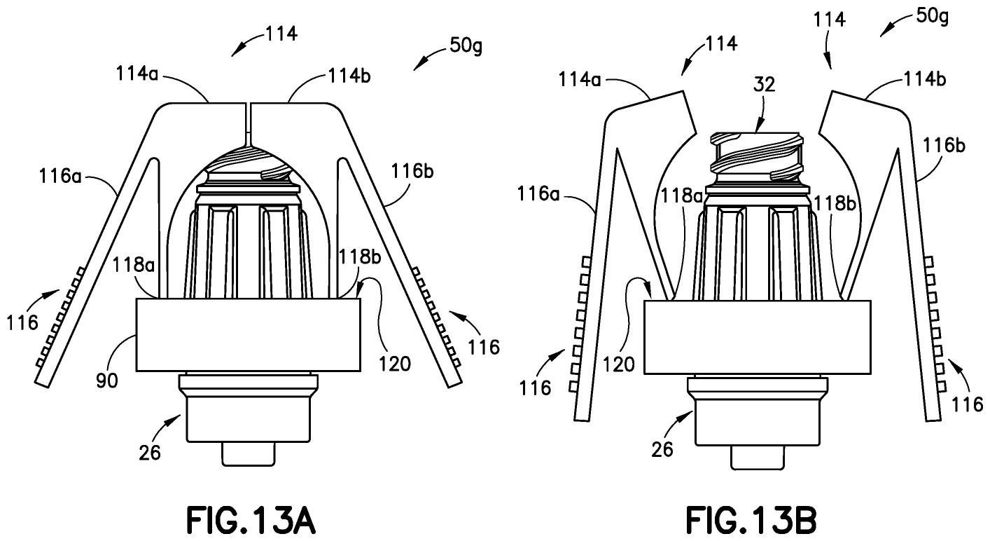

Referring now to FIGS. 13A and 13B, a protective cap 50g is shown in accordance with another embodiment of the disclosure. The collar member 90 of protective cap 50g may be identical to the collar member 90 included in the protective caps 50c, 50f of FIGS. 9-11 and 12, but protective cap 50g includes cover portions 114a, 114b (of a cover member 114) and levers 116a, 116b (of a lever member 116) that are arranged and operate to pivot the cover portions 114a, 114b in a different manner from the cover portions 92a, 92b and levers 94a, 94b of FIGS. 9-11 and 12.

As shown in FIGS. 13A and 13B, protective cap 50g includes a first hinge 118a and a second hinge 118b positioned on a top side 120 of the collar member 90, adjacent to opposing first and second sides of the collar member 90. The first hinge 118a connects the first cover portion 114a to the collar member 90 and the second hinge 118b connects the second cover portion 114b to the collar member 90, such that the first cover portion 114a and the second cover portion 114b pivot relative to the collar member 90 via the first hinge 118a and the second hinge 118b, respectively. The first and second hinges 118a, 118b thus enable the cover portions 114a, 114b to pivot from a closed or “first” position, where the cover portions 114a, 114b substantially enclose or cover the female luer connection 32, to an open or “second” position, where the female luer connection 32 is uncovered and accessible for connecting a male luer connection of another component/device thereto.

The first and second levers 116a, 116b are attached to the respective first and second cover portions 114a, 114b and are configured so as to be engageable by a user to move the levers 116a, 116b from a non-actuated position to an actuated position. In the embodiment of FIGS. 13A and 13B, the levers 116a, 116b are attached to the respective first and second cover portions 114a, 114b so as to extend downwardly and outwardly therefrom. A far end of each lever 116a, 116b is provided as a free end that may be pushed radially inward by a user, via a single hand actuation, to move the levers 116a, 116b from their non-actuated position to their actuated position. Movement of the levers 116a, 116b from their non-actuated position to their actuated position causes a corresponding movement of the cover portions 114a, 114b from their first position to their second position, thereby uncovering the female luer connection 32 of the needle-free connector 26.

Beneficially, embodiments of the invention thus provide a protective cap attachable to a needle-free connector that maintains the cleanliness thereof. The protective cap is adaptable to different sizes/shapes of needle-free connectors and may be easily handled or actuated by a clinician when preparing to attach another component/device to the needle-free connector.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A protective cap engageable with a needle-free connector of an intravenous (IV) catheter assembly, the protective cap comprising:
a collar member defining a central opening within which a portion of the needle-free connector may be received;
a cover member attached to the collar member so as to be pivotable or slideable relative thereto, the cover member moveable between a first position where an end connection of the needle-free connector is covered by the cover member and a second position where the end connection of the needle-free connector is uncovered from the cover member; and
a lever member attached to the cover member and engageable by a user to move the lever member from a non-actuated position to an actuated position;
wherein with the lever member in the non-actuated position, the cover member is in the first position; and
wherein with the lever member in the actuated position, the cover member is in the second position.

2. The cap of claim 1, wherein the collar member comprises a friction pad positioned on an inner surface of the collar member configured to secure the collar member on the portion of the needle-free connector.

3. The cap of claim 1, wherein the collar member comprises:
a first collar half including a buckle receptacle; and
a second collar half including a buckle stem;
wherein the first collar half is joinable to the second collar half via insertion of the buckle stem into the buckle receptacle, with positioning of the buckle stem relative to the buckle receptacle being adjustable, such that a diameter of the central opening of the collar member is also adjustable.

4. The cap of claim 1, comprising a hinge connecting the cover member to the collar member, the hinge positioned at a top edge of the collar member, with the cover member pivoting relative to the collar member via the hinge to move between the first position and the second position.

5. The cap of claim 4, wherein the lever member comprises a downward oriented lever arranged approximately parallel to an outer surface of the collar member, the lever member being pullable downward to move the lever member from the non-actuated position to the actuated position, thereby pivoting the cover member from the first position to the second position.

6. The cap of claim 4, wherein the lever member comprises a generally horizontally oriented lever extending radially outward from the cover member, the lever member being pushable downward to move the lever member from the non-actuated position to the actuated position, thereby pivoting the cover member from the first position to the second position.

7. The cap of claim 4, wherein the lever member comprises a V-shaped push lever linkage attached to the cover member at a first linkage end and to the collar member at a second linkage end, the lever member being pressable radially inward to move the lever member from the non-actuated position to the actuated position, thereby pivoting the cover member from the first position to the second position.

8. The cap of claim 6, wherein the hinge is biased to return the cover member from the second position to the first position, upon termination of a pushing force on the lever member.

9. The cap of claim 1, wherein the collar member comprises a pair of restraint arms extending upwardly past a tope edge of the collar member, each restraint arm of the pair of restraint arms comprising a hook member at a distal end thereof, and wherein the cover member comprises tracks formed thereon with which the hook member of each restraint arm is slidingly engaged.

10. The cap of claim 9, wherein the lever member comprises an angled lever extending downwardly and outwardly from the cover member, the lever member being pressable radially inward toward the collar member by a user to move the lever member from the non-actuated position to the actuated position, thereby sliding the cover member from the first position to the second position, via sliding of the hook member of each restraint arm along the tracks.

11. The cap of claim 1, wherein the cover member comprises a first cover portion and a second cover portion and wherein the lever member comprises a first lever and a second lever, with the first lever attached to the first cover portion and the second lever attached to the second cover portion.

12. The cap of claim 11, comprising a first fulcrum feature positioned on a first side of the collar member and a second fulcrum feature positioned on a second side of the collar member, with the first lever attached to the first cover portion at the first fulcrum feature and the second lever attached to the second cover portion at the second fulcrum feature, such that the first lever and the first cover portion pivot about the first fulcrum feature and the second lever and the second cover portion pivot about the second fulcrum feature.

13. The cap of claim 12, comprising a first bi-stable hinge connecting the first fulcrum feature to the collar member and a second bi-stable hinge connecting the second fulcrum feature to the collar member, the first bi-stable hinge and the second bi-stable hinge positioned on opposing first and second sides of the collar member, with the first cover portion and the second cover portion pivoting relative to the collar member via the first bi-stable hinge and the second bi-stable hinge, respectively, to move between the first position and the second position.

14. The cap of claim 11, comprising a first hinge connecting the first cover portion to the collar member and a second hinge connecting the second cover portion to the collar member, the first hinge and the second hinge positioned on a top side of the collar member, with the first cover portion and the second cover portion pivoting relative to the collar member via the first hinge and the second hinge, respectively, to move between the first position and the second position.

15. The cap of claim 11, wherein each of the first lever and the second lever is pressable radially inward to move the first lever and the second lever from the non-actuated position to the actuated position, thereby pivoting each of the first cover portion and the second cover portion from the first position to the second position.

16. The cap of claim 15, wherein the first bi-stable hinge and the second bi-stable hinge are configured to hold the first cover portion and the second cover portion in the second position upon termination of a radially inward directed pushing force on the first lever and the second lever.

17. The cap of claim 1, wherein the lever member is engageable by a single hand of the user to move the lever member from the non-actuated position to the actuated position.

18. An intravenous (IV) catheter assembly comprising:
a catheter adapter;
a catheter coupled to the catheter adapter and extending out distally therefrom, so as to be positionable intravenously within a patient;
an extension line coupled to the catheter adapter and extending out proximally therefrom;
a luer hub positioned at a proximal end of the extension line, the luer hub in fluid communication with the catheter through the extension line and the catheter adapter;
a needle-free connector comprising a distal connector end and a proximal connector end, the distal connector end coupled to the luer hub; and
a protective cap having:
a collar member defining a central opening within which a portion of the needle-free connector may be received,
a cover member attached to the collar member so as to be pivotable or slideable relative thereto, the cover member moveable between a first position where an end connection of the needle-free connector is covered by the cover member and a second position where the end connection of the needle-free connector is uncovered from the cover member, and
a lever member attached to the cover member and engageable by a user to move the lever member from a non-actuated position to an actuated position;
wherein with the lever member in the non-actuated position, the cover member is in the first position,
wherein with the lever member in the actuated position, the cover member is in the second position; and
wherein the protective cap is coupled to the proximal connector end of the needle-free connector.

19. The IV catheter assembly of claim 18, wherein the proximal connector end of the needle-free connector comprises a female luer connection comprising a tapered cavity and a threaded outer surface.

20. The IV catheter assembly of claim 19, wherein the collar member is positioned about the needle-free connector at a location thereon distal from the threaded outer surface; and
wherein the tapered cavity and threaded outer surface are uncovered from the protective cap when the protective cap is in the first position and are covered by the protective cap when the protective cap is in the second position.
